(19) United States
(12) Patent Application Publication
(10) Pub. No.: US 2014/0125948 A1
(43) Pub. Date: May 8, 2014

(54) Positioning Unit And Observation Device

(71) Applicants: Carsten Feiertag, Hungen (DE); Guenter Pfeiffer, Gladenbach (DE); Rainer Kirchhuebel, Asslar (DE)

(72) Inventors: Carsten Feiertag, Hungen (DE); Guenter Pfeiffer, Gladenbach (DE); Rainer Kirchhuebel, Asslar (DE)

(21) Appl. No.: 13/667,488

(22) Filed: Nov. 2, 2012

(51) Int. Cl. A61B 3/12; A61B 3/00

(52) U.S. Cl. CPC A61B 3/12; A61B 3/0075
USPC 351/205; 351/245

(57) ABSTRACT

A positioning unit for positioning an optical unit in an optical path of a microscope between a microscope lens and in front of an eye to be examined includes a connection device and a positioning device. The connection device couples the positioning unit to the microscope. The positioning device moves the optical element relative to the microscope in a longitudinal direction of the optical path. The positioning unit is predominantly formed from aluminum free metal.

### CROSS REFERENCE TO RELATED APPLICATION

Not applicable.

### STATEMENT REGARDING FEDERALLY SPONSORED RESEARCH OR DEVELOPMENT

Not applicable.

### FIELD OF THE INVENTION

The invention relates to a positioning unit for positioning an optical unit, comprising at least one optical element, in an optical path of a microscope between the lens of the microscope and in front of an eye to be examined, wherein the positioning unit comprises a connection device, by means of which the positioning unit can be coupled to the microscope and wherein the positioning unit comprises a positioning device, by means of which the optical element can be moved relative to the microscope in a longitudinal direction of the optical path, wherein the positioning unit is formed predominantly of metal.

### BACKGROUND OF THE INVENTION

Microscopes for performing eye operations are regularly used for operations in a front area of an eye. If such operations are to be performed in a rear area of an eye, it is necessary to supplement the microscope with an examination apparatus, which makes it possible to focus on said area of the eye. Such examination apparatuses comprise at least one wide angle lens or an ophthalmoscopic lens for a wide angle view of the respective rear part of the eye, wherein the ophthalmoscopic lens provides an intermediate image in an optical path in front of a lens of the microscope, on which the microscope can be focused. For focusing on the intermediate image, a shortening of a length of the optical path of the microscope is required, which can be achieved by means of the corresponding adjusting facilities on the microscope. However, since during an eye operation it is necessary to switch between different ways of examination with and without an ophthalmoscopic lens, such an adjustment of the microscope is inconvenient. Therefore, a reducing lens can be provided in the optical path in front of the lens, which serves for shortening the optical path of the microscope and which is used together with the ophthalmoscopic lens. The two lenses are held by a positioning unit of the examination apparatus, which is mounted directly on the microscope, and can be positioned in the optical path as needed without a substantial adjustment of the microscope being required during an operation. The positioning unit typically comprises a connection device, by means of which the positioning unit can be coupled to the microscope. Further, the positioning unit is formed in such a manner that the respective lenses can simply be pivoted or slid into the optical path and out again.

In order to achieve an adjustment as precise as possible of the intermediate image of the ophthalmoscopic lens to a focal length of the microscope lens, at least one of the lenses can be formed adjustable lengthwise of the optical path of the microscope. In known examination apparatus, a linear guide is formed, for example, on the positioning unit for a longitudinally slidable adjustment of the lens, wherein the lens can be moved by means of an adjustment dial with a screw drive. In order to avoid an accidental collision of the ophthalmoscopic lens and the eye and to avoid possible eye damage during an eye operation, the positioning unit is formed in such a manner that the ophthalmoscopic lens is substantially movable without resistance in the direction of the lens of the microscope, which means that it can retreat in the case of a collision with the eye. For example, this is achieved by means of a second linear guide, which also provides for a longitudinal slide of the ophthalmoscopic lens.

Apart from the afore-described mechanical and optical requirements, it is important that the examination apparatus or the positioning unit be substantially sterile during an operation so as to prevent a possible infection of the eye, for example, with germs. A risk of infection arises in particular from the examination apparatus being put relatively close to the respective eye during an operation. Therefore, it is common practice to clean the respective examination apparatus or positioning unit prior to an operation, for example, by steam sterilization. For repeated sterilization to be possible, it is essentially necessary to form all components of the examination apparatus or positioning unit from metal or glass, excluding all seals made of elastic materials, such as rubber. Other materials, such as plastics, have proven unsuitable for repeated sterilization. The linear guides and the screw drive require an accurate form for ensuring certain fits so that only components made of metal are an option here as well. In order to prevent water from entering the guides during sterilization they can be provided with rubber seals or seals from other elastic materials. The metal components are typically made of aluminum or an aluminum alloy. Aluminum can be machined particularly well and has a low weight, which is advantageous for a handling of the examination apparatus. The components made of aluminum are further protected against corrosion by an anodized coating (Eloxal).

In particular if an examination apparatus or a positioning unit is sterilized relatively often, the problem arises that an aluminum surface is corroded and destroyed due to chemical reactions. This is caused by alkaline detergents, which are used for pre-cleaning the examination apparatus or positioning unit prior to steam sterilization. Therefore, the examination apparatus or positioning unit is only suited and, thus, usable for a limited number of sterilization cycles due to a destruction of the surface of the components made of aluminum. Corroded surfaces or components made of aluminum can no longer be cleaned in the necessary manner and, thus, can no longer be used for eye operations.

### SUMMARY OF THE INVENTION

Therefore, it is the object of the present invention, to provide a positioning unit and an examination apparatus whose usability is not influenced by the common use of detergents.

This object is attained in one embodiment by a positioning unit for positioning an optical unit, which comprises at least one optical element, in the optical path of a microscope between a lens of the microscope and in front of an eye to be examined comprises a connecting device, by means of which the positioning unit can be coupled to the microscope, wherein the positioning unit further comprises a positioning device, by means of which the optical element can be moved relative to the microscope in the longitudinal direction of the optical path, wherein the positioning unit is predominantly formed from metal, and wherein at least the positioning device is formed free of aluminum.

Consequently, the positioning unit is made of metal, except for the necessary seals, but no aluminum or aluminum alloy is used for the components of the positioning unit or the positioning device. Thus, it is made sure that the destruction of a component surface by alkaline cleaning, as observed in aluminum, cannot occur. The positioning unit can then be subjected almost arbitrarily often to alkaline cleaning or sterilization. Preferably then, metals are used for forming the positioning unit which are particularly well suited for alkaline cleaning.

Advantageously, the positioning unit can be formed from titanium and steel or titanium and ceramics. Thus, it can be made sure that none of the used materials are corroded by alkaline cleaning. Further, titanium is particularly well suited as a material here because titanium has a relatively low density and, thus, does not substantially increase a weight of the positioning in the case of an additional use of steel. Also, individual components of the positioning unit can be formed from a ceramic material. Ceramic materials can have a low density and a good chemical durability as well. Alternatively, it is of course possible as well to form the positioning unit from titanium, steel and ceramics. The positioning unit can be formed entirely from the aforementioned materials.

It is particularly advantageous if components of the positioning unit which form a slide surface combination and are moveable in relation to one another are of a material combination of titanium/steel or of titanium/ceramics. Material combinations can be used which have a particularly favorable friction coefficient. Then, where appropriate, even an otherwise common lubrication of the slide surface combination can be omitted. Titanium in particular has a low friction coefficient.

Further, stainless steel can be used for steel and a titanium alloy for titanium. These materials can then be adapted even better to their use. Therein, the stainless steel and the titanium alloy can also contain aluminum as an alloy addition.

The positioning device can comprise an adjusting facility, by means of which a position of the optical element in the longitudinal direction of the optical path can be adjusted. A mobility of the optical element in the longitudinal direction of the optical path relative to the microscope allows for an adjustment of the optical unit to the eye to be observed and/or an adjustment of the optical path of the microscope to an intermediate image within the optical path without any adjustments on the microscope becoming necessary to this effect.

Therein, the adjusting facility can be formed from a threaded rod, a guiding rod and from a holding element connected to the threaded rod and the guiding rod, wherein the threaded rod and the guiding rod can be formed from steel and the holding element can be formed from titanium. In sections, he holding element can comprise threads, which can engage the threaded rod. Thus, by turning the threaded rod, it is possible to move the holding element along the guide rod and, thus, relative to the microscope in the longitudinal direction of the optical path and to precisely adjust it. For this, the holding element does not even have to surround the threaded rod in the manner of a nut. Further, a dial can be formed on the threaded rod for manually working it. An adjustment or a positioning of the optical element can, for example, take place manually by an operating person.

For protecting an eye from an unintentional collision with the optical element, the positioning unit can comprise a safety facility, which allows for a lose movement of the optical element, when a force in the direction of the microscope is applied to the optical element. This means, the positioning device or the safety facility can be formed in such a manner that, when a force is applied to the optical element, caused, for example, by a collision with the respective eye, the optical element can be moved substantially without resistance in the direction of the lens. The safety facility can be formed in such a manner that the positioning device and the optical unit hold the optical element in a lower position in the vicinity of the eye by means of their respective own weight. If then a force is applied to the optical element in the direction of the microscope, a mere weight force of the optical unit or the positioning device have to be overcome in order to move the optical element. In order to eliminate an undesired movement of the optical element, a spring can be provided on the safety facility for stabilizing the optical element, said spring applying an additional force in the direction of the eye.

For example, the safety facility can be formed from a holding rod guided in a holding element, wherein the holding rod can be formed from steel or ceramics and the holding element can be formed from titanium. On a lower end of the holding rod an optical element can then be attached. The holding element can be formed as one piece or also be a component of an adjusting facility.

Further, the positioning device can comprise a holding facility, by means of which the optical element can be moved in and out of the optical path and by means of which the positioning device can be detachably connected without tools to the connection device. The connection device can be formed directly rigidly connectable to the microscope, wherein the connection device can be connected to the holder device, for example in the manner of a plug connection, without the help of any additional tools.

Further, the holder device can be formed in such a manner that the optical element can be pivoted or slid into the optical path. Preferably, the optical element together with the positioning unit can be pivoted around an axis running transverse to the optical path. Thus, it can be made sure that during an operation the positioning unit and the optical unit do not limit or obstruct a view in a moving space of the operator outside of the respective eye. Also, it is thereby made possible to simply pivot the optical element in and out of the optical path as needed.

The holding facility can be formed from a holding arm for holding an adjusting facility and from a fastening element for the connection to the connection device, wherein the holding arm can be formed from titanium and the fastening element can be formed from steel. The fastening element can then be formed in such a manner that it can be particularly easily connected to or, for example, locked on the connection device.

Further, the fastening element can comprise an axis which forms a swivel joint with the holding arm. By means of the swivel joint the adjusting facility can be pivotable relative to the connection device. For forming such a swivel joint, additional components are not necessarily needed. However, the axis can also be formed as a single component made of steel, wherein the holding arm made of titanium can then simply be plugged onto the axis.

For a simpler handling, the swivel joint can comprise at least one locking facility, by means of which the optical element can be locked in a usage position in the optical path and/or in a non-usage position outside of the optical path. The locking facility can be formed in such a manner that between the holding arm and the fastening element a locking nib in locking depressions is formed for the locking nib to engage. The locking nib and the locking depressions can be formed on the holding arm or the fastening element, respectively. The locking depressions can then be arranged such that, in the usage position and the non-usage position, the locking nib engages a locking depression, respectively, and, thus, a locking of the optical element or the adjusting facility is possible.

Apart from that, the holding arm of a holding socket can comprise a reducing lens of the optical unit, wherein the holding socket can be formed from steel. The holding socket can then simply be inserted into the holding arm made of titanium. Thus, it is possible to also simply exchange the reducing lens with a reducing lens with other optical properties.

An examination apparatus according to the invention comprises a positioning unit, such as described herein, wherein the optical unit comprises at least one optical element which is formed as an ophthalmoscopic lens, which serves for examining an ocular fundus. With regard to the advantages of the examination apparatus according to the invention, reference is made to the preceding descriptions of advantages of the positioning unit.

As a further optical element, an optical unit can comprise a reducing lens, which serves for adjusting the optical path. Further, additional optical units for image inversion and/or an exchange of two optical paths can be provided.

In an embodiment, the examination apparatus can comprise an electric drive device, which serves for moving the optical element in the longitudinal direction of the optical path. The electric drive device can be formed in the manner of an electric motor and can drive a possibly present adjusting facility of the positioning unit. The electric motor can be accommodated in a separate waterproof housing and be connected to the positioning unit via a gear or another kind of coupling. In this way, it is also possible to sterilize the electric motor or the electric drive device. Preferably, the housing is formed from titanium.

Further advantageous embodiments of the examination apparatus arise from features of embodiments of the invention described herein.

### BRIEF DESCRIPTION OF THE DRAWINGS

In the following, preferred embodiments of the invention are described in more details with reference to the accompanying drawings.

FIG. 1 shows an examination apparatus incorporating the present invention in a perspective view;

FIG. 2 shows the examination apparatus of FIG. 1 with a trapezoidal extension;

FIG. 3 shows the examination apparatus of FIG. 2 with an electric motor drive; and

FIG. 4 shows a connection device for use with the examination apparatus of FIG. 1 in a perspective view.

### DETAILED DESCRIPTION OF THE EXAMPLE EMBODIMENTS

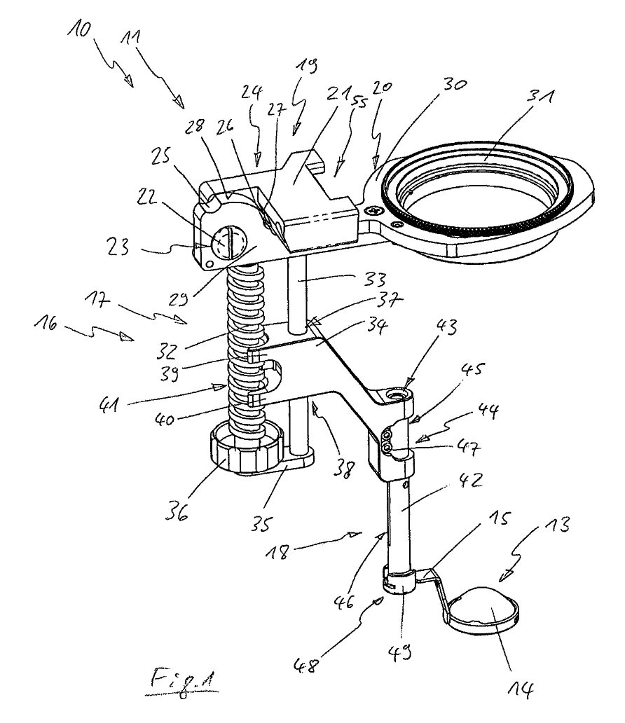

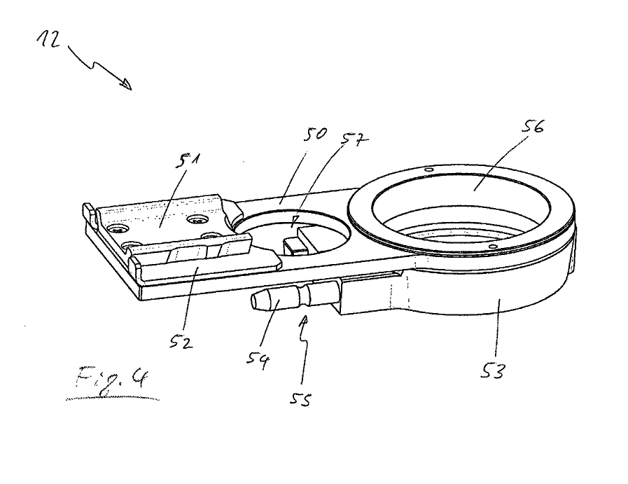

FIG. 1 shows an examination apparatus 10 with a positioning unit 11, however, without a connection device. An associated connection device 12 can be taken from FIG. 4.

The examination apparatus 10 comprises, apart from the connection device 12 and the positioning unit 11, an optical unit 13, of which here an ophthalmoscopic lens 14 on a holding arm 15 is depicted. The positioning unit 11 further comprises a positioning device 16 with an adjusting facility 17, a safety facility 18 and a holding facility 19. The holding facility 19 is formed from a holding arm 20 made of titanium for holding the adjusting facility 17 and a fastening element 21 for the connection to the connection device 12. The fastening element 21 is made of steel and comprises an axis 22, also made of steel, which, together with the holding arm 20, forms a swivel joint 23.

The swivel joint 23 comprises a locking facility, by means of which the ophthalmoscopic lens 14 can be locked in a not further illustrated usage position within a not further illustrated optic path or in a non-usage position outside of the optic path. The locking facility 24 comprises two locking depressions 25 and 26, the holding arm 20 and a locking nib 27 on the locking element 21. The locking nib 27 is pressed onto a bend-shaped abutment surface 28 on the holding arm 20 by a spring not visible here, which is arranged within the fastening element 21.

The holding arm 20 is formed from a first metal sheet 29 and a second metal sheet 30 made of titanium, which are screwed together. In the second metal sheet 30, a holding socket 31 made of steel is inserted for a reducing lens not illustrated here. On the first metal sheet 29, a threaded rod 32 of the adjusting facility 17 is rotatably attached. Further, on the first metal sheet 29, a guiding rod 33 of the adjusting facility 17 is rigidly mounted. The threaded rod 32 and the guiding rod 33 are formed from steel. The adjusting facility 17 further comprises a holding element 34 and a bearing element 35 made of titanium. The bearing element 35 connects the threaded rod 32 to the guiding rod 33, wherein the threaded rod 32 is mounted rotatably in the bearing element 35. Apart from that, a working dial 36 is arranged on the threaded rod 32, by means of which the threaded rod 32 can be manually rotated. The holding element 34 comprises a passage bore 37, into which the guiding rod 33 is inserted so that here, between the guiding rod 33 and the holding element 34, a linear guide 38 is formed. Further, an upper and a lower engagement nib 39 and 40 are formed on the holding element 34, which comprise threads not visible here for the corresponding engagement into a thread 41 of the threaded rod 32. The engagement nibs 39 and 40 therein only semi-surround the threaded rod 32. Thus, a rotating of the threaded rod 32 leads to a movement of the holding element 34 in the longitudinal direction of the guiding rod 33 and, thus, in the longitudinal direction of the optic path.

Further, on the holding element 34, a holding rod 42 is mounted, which, together with the holding element 34, forms the safety facility 18. The holding rod 42 is made of steel and is movably inserted into a passage bore 43 in the holding element 34. Thus, a linear guide is formed between the holding element 34 and the holding rod 42 here as well. On the holding element 34 in the area of the passage bore 43, a recess 45 is further formed, which opens the passage bore 43 in a sectional manner. On the holding rod 42, a guiding groove 46 is formed, into which a guide element 47 engages on the holding element 34 and, thus, limits a longitudinal and rotating movement of the holding rod 42 relative to the holding element 34. On a lower end 48 of the holding rod 42, a connection element 49 is arranged, into which the holding arm 15 is inserted.

The connecting device 12 shown in FIG. 4 consists of a metal sheet 50 made of titanium, on which a guiding element 51 and an abutment metal sheet 52 made of steel are rigidly mounted on a not illustrated microscope for the adaption of the connecting device 12. Further, a holding element 53 made of titanium is fastened to the metal sheet 50, which holding element 53 comprises a locking pin 54 made of steel for the connection to the holding element 34. Between the holding element 53 and the holding element 34, thus, a corresponding plug connection 55 is formed. Further, a ring 56 made of steel is inserted into the metal sheet 50 and the holding element 53, which ring can surround a lens of a microscope not illustrated here. For reducing the weight, a recess 57 is formed in the metal sheet. 50.

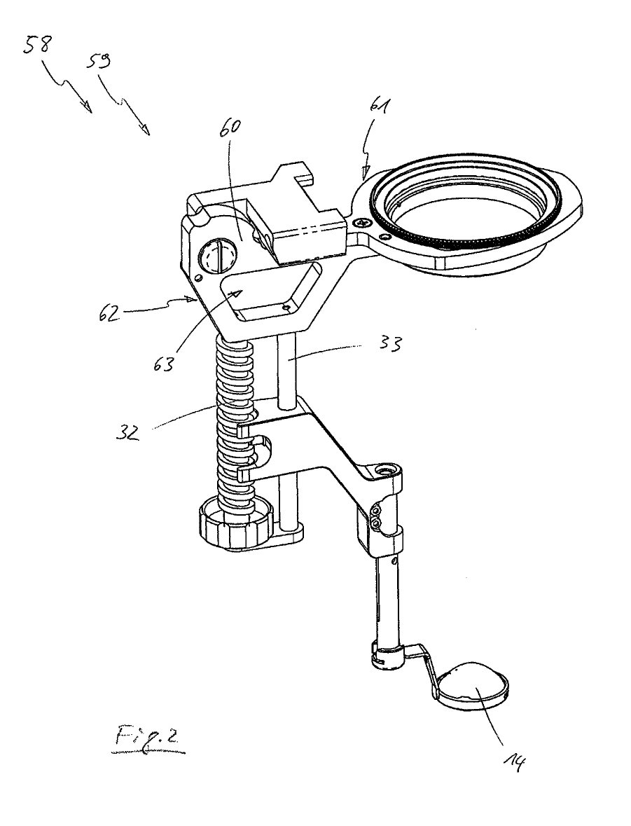

FIG. 2 shows an examination apparatus 58 with a positioning unit 59 without a connection device. The examination apparatus 58 or the positioning unit 59 is connectable to the connection device 12 from FIG. 4. In contrast to the positioning unit 11 from FIG. 1, in the positioning unit 59 a first metal sheet 60 of a holding arm 61 of the positioning unit 59 with a trapezoidal extension 62 is formed in order to increase a distance of the ophthalmoscopic lens 40 to a not illustrated lens of the microscope. Further, in the first metal sheet 60, a recess 63 is formed in order to achieve a weight reduction.

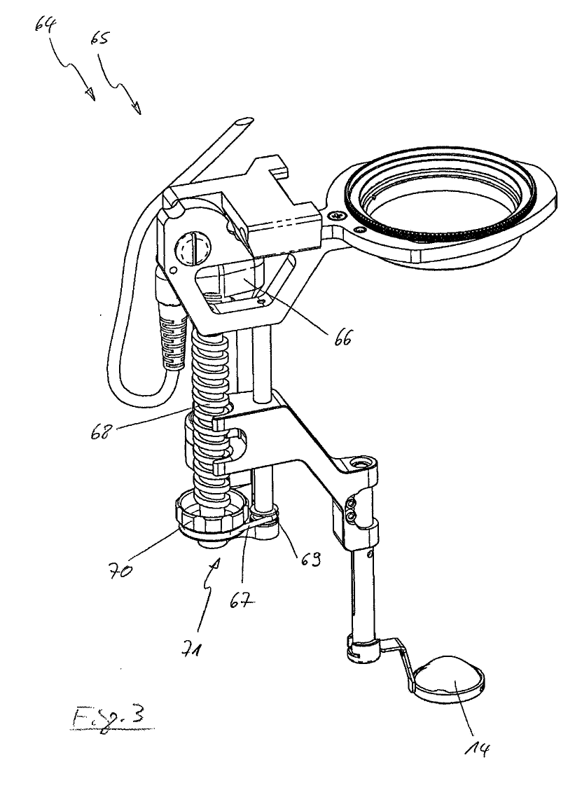

FIG. 3 shows an embodiment of an examination apparatus 64 with a positioning unit 65 and without a connection device, wherein here the examination apparatus 64 or the positioning unit 65 is connectable to the connection device 12 from FIG. 4 as well. In contrast to the examination apparatus or positioning device illustrated in FIG. 2, the examination device 64 comprises an electric motor drive 66, which is adapted to the positioning unit 65. The drive 66 is connected to a threaded rod 68 and a return pulley 69 via a belt 67. The return pulley 69 is rotatably mounted on the guiding rod 33 and a working dial 70 is formed in sections on the threaded rod 68 in the manner of a belt pulley so that the belt 67 can drive the working dial 70 and rotate the threaded rod 68. The drive 66 together with the belt 67 and the working dial 70 thus forms a belt drive 71.

1. A positioning unit for positioning an optical unit in an optical path of a microscope between a microscope lens and in front an eye to be examined, said optical unit including at least one optical element, wherein the positioning unit comprises:
a connection device adapted to couple the positioning unit to a microscope having a substantially vertical optical path; and
a positioning device moving an optical element of an optical unit relative to the microscope in a longitudinal direction of the optical path, wherein the positioning unit is formed predominantly from metal, said metal being free of aluminum.

2. The positioning unit according to claim 1, in which the positioning unit is formed from materials selected from a group consisting of a combination of titanium and steel and a combination of titanium and ceramics.

3. The positioning unit according to claim 1, in which components of the positioning unit form a sliding surface combination and are movable relative to one another, said components being formed from materials including a material combination selected from a group consisting of titanium/steel and titanium/ceramics.

4. The positioning unit according to claim 2, in which said steel is a stainless steel and said titanium is a titanium alloy.

5. The positioning unit according to claim 1, in which the positioning device includes an adjusting facility adjusting a position of the optical element in the longitudinal direction of the optical path.

6. The positioning unit according to claim 5, in which the adjusting facility is formed from a threaded rod, a guiding rod and a holding element, said holding rod being connected to the threaded rod and the guiding rod, wherein the threaded rod and the guiding rod are formed from steel and the holding element is formed from titanium.

7. The positioning unit according to claim 1, in which the positioning device includes a safety facility, said safety facility allowing movement of the optical element when a force is applied to the optical element toward the microscope.

8. The positioning unit according to claim 7, in which the safety facility is formed from a holding rod guided in a holding element, wherein the holding rod is formed from at least one of steel and ceramics and the holding element is formed from titanium.

9. The positioning unit according to claim 1, in which the positioning device includes a holding facility moving the optical element in and out of the optical path, said holding facility, detachably connecting the positioning unit without tools to the connection device.

10. The positioning unit according to claim 9, in which the holding facility is formed from a holding arm holding an adjusting facility and a fastening element connecting the holding arm to the connection device, wherein the holding arm is formed from titanium and the fastening element is formed from steel.

11. The positioning unit according to claim 10, in which the fastening element includes an axis which, together with the holding arm, forms a swivel joint.

12. The positioning unit according to claim 11, in which the swivel joint comprises at least one locking facility locking the optical element in a usage position in at least one of the optical path and a non-usage position outside of the optical path.

13. The positioning unit according to claim 10, in which the holding arm includes a holding socket for a reducing lens of the optical unit, wherein the holding socket is formed from steel.

14. An examination apparatus with a positioning unit according to claim 1 and at least one optical unit, wherein the optical unit comprises:
at least one optical element formed as an ophthalmoscopic lens usable for examining an ocular fundus.

15. The examination apparatus according to claim 14, in which the optical unit includes a reducing lens as a further optical element adjusting the optical path.

16. The examination apparatus according to claim 14, in which the examination apparatus includes an electric drive device moving the optical element in the longitudinal direction of the optical path.
